(19) United States
(12) Patent Application Publication
(10) Pub. No.: US 2009/0185655 A1
(43) Pub. Date: Jul. 23, 2009

(54) COMPUTED TOMOGRAPHY METHOD

(75) Inventors: Peter Koken, Hamburg (DE); Andy Ziegler, Hamburg (DE); Michael Grass, Buchholz in der Nordheide (DE); Thomas Köhler, Norderstedt (DE); Roland Proksa, Hamburg (DE)

Correspondence Address: PHILIPS INTELLECTUAL PROPERTY & STANDARDS, P. O. Box 3001, BRIARCLIFF MANOR, NY, 10510, US

(73) Assignee: KONINKLIJKE PHILIPS ELECTRONICS N.V., Eindhoven (NL)

(21) Appl. No.: 11/575,662

(22) PCT Filed: Sep. 23, 2005
(86) PCT No.: PCT/IB05/53154

§ 371 Date: Mar. 21, 2007

(30) Foreign Application Priority Data
Oct. 6, 2004 (EP) 04104892.7

(51) Int. Cl. A61B 6/00; G21K 1/00

(52) U.S. Cl. 378/11; 378/154

(57) ABSTRACT

A computed tomography method and apparatus are provided wherein a radiation source moves circularly relative to an examination zone about an axis of rotation (14). The radiation source produces a cone beam of x-rays and the focal point of this cone beam is switched between at least two positions (23a, 23b) spaced apart from each other and arranged on a line parallel to the axis of rotation to enlarge the reconstructable examination zone parallel to the axis of rotation. Preferably, the image of the examination zone is reconstructed using an iterative reconstruction method, in particular an algebraic reconstruction method or a maximum likelihood method.

The invention relates to a computed tomography method in which a radiation source moves relative to an examination zone circularly about an axis of rotation. The radiation source emits a conical radiation beam traversing the examination zone, measured values are acquired by a detector unit during the relative motion and an image of the examination zone is reconstructed using the measured values.

The invention also relates to a computed tomography apparatus for carrying out the computed tomography method as well as to a computer program for controlling the computed tomography apparatus.

The dimension of the reconstructable examination zone parallel to the axis of rotation is limited by the cone angle of the conical radiation beam. A smaller cone angle leads to a smaller dimension of the reconstructable examination zone parallel to the axis of rotation, whereas a larger cone angle leads to a larger dimension of the reconstructable examination zone parallel to the axis of rotation. The cone angle is the angle enclosed by a ray from the radiation source to an outermost edge of a detecting surface of the detector unit in a direction parallel to the axis of rotation and a plane in which the radiation source rotates relative to the examination zone. Thus, the cone angle is defined by the distance between the radiation source and the detecting surface of the detector unit and the dimension of the detecting surface parallel to the axis of rotation.

Because of the limited dimension of the detecting surface in the direction parallel to the axis of rotation, the cone angle of known computed tomography apparatus and thus the dimension of the reconstructable examination zone parallel to the axis of rotation is too small for many applications, e.g. a heart of a human patient is too large to be situated completely in the reconstructable examination zone.

It is therefore an object of the invention to provide a computed tomography method which has an enlarged reconstructable examination zone parallel to the axis of rotation.

This object is achieved by means of a computed tomography method in accordance with the invention comprising the steps of:
generating a circular relative motion between an examination zone and a radiation source about an axis of rotation,
generating a conical radiation beam using the radiation source, wherein the conical radiation beam is emitted from an emitting area of the radiation source, wherein the conical radiation beam traverses the examination zone and wherein the position of the emitting area is moved parallel to the axis of rotation during the relative motion,
acquiring measured values using a detector unit during the relative motion, wherein the measured values depend on the intensity of the conical radiation beam after traversing the examination zone,
switching the position of the emitting area between at least two positions (23a, 23b) spaced apart from each other and arranged on a line parallel (27) to the axis of rotation (14) during the relative motion,
reconstructing an image of the examination zone using the measured values.

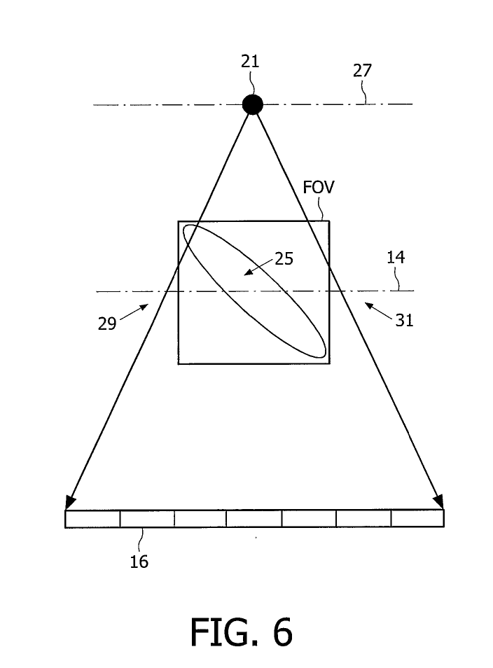

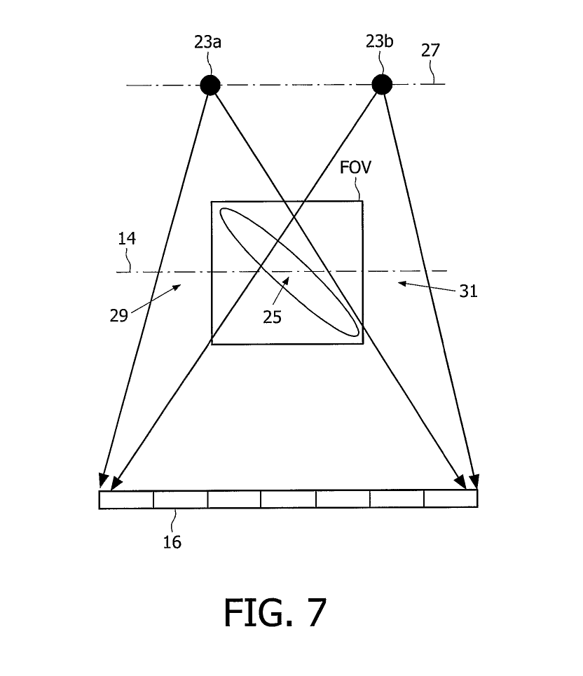

The movement of the emitting area parallel to the axis of rotation during the relative motion leads to an enlargement of the dimension of the reconstructable examination zone parallel to the axis of rotation. This is described in more detail with reference to FIGS. 6 and 7 further below. Thus, compared to known computed tomography methods larger objects can be reconstructed using a circular movement of the radiation source relative to the examination zone.

The position of the emitting area is switched between at least two positions spaced apart from each other and arranged on a line parallel to the axis of rotation, i.e. the emitting area is not continuously moved parallel to the axis of rotation, but the emitting area is positioned at one of at least two locations and the radiation source switches the position of the emitting area from one location to another location during acquisition. If the radiation source switches the position of the emitting area from a first location to a second location having a certain distance, the enlargement of the reconstructable examination zone is the same as if the radiation source would move the emitting area continuously along the same distance, but a different sampling of the views would result yielding a further improved image quality.

When the radiation source is situated in a certain angular range of the circle on which the radiation source moves relative to the examination zone, only measured values might be acquired, while the emitting area is positioned at the same location within the radiation source. While, when the radiation source is situated in another angular range of the circle, only measured values might be acquired, while the emitting area is positioned at another location within the radiation source. Thus, the angular positions of the radiation source, while the emitting area is positioned at a certain location within the radiation source, might be distributed quite non-uniformly, so that the quality of an reconstructed image of the examination zone might be poor.

The embodiment in accordance with claim 2 ensures a more uniform distribution of the angular position of the radiation source, while the emitting area is positioned at a certain location, resulting in an improved image quality.

The iterative reconstruction method according to claim 3 leads to a more homogenous image quality compared to other known reconstruction methods like filtered back projection.

A computed tomography apparatus for carrying out the computed tomography method in accordance with the invention is disclosed in claim 4. The embodiments disclosed in claims 5 and 6 result in a reduction of artifacts caused by scattering. Claim 7 defines a computer program for controlling the computed tomography apparatus as disclosed in claim 4.

The invention will be described in detail hereinafter with reference to the drawings, wherein

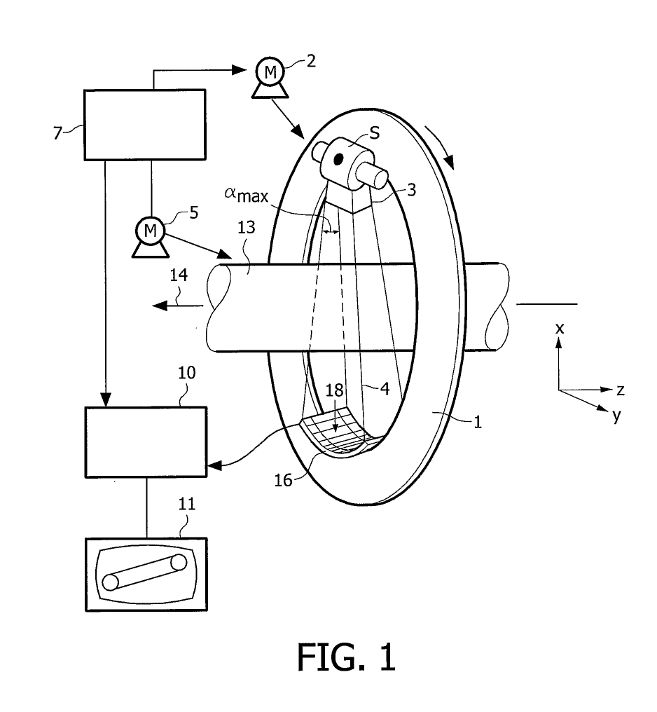

FIG. 1 shows a computed tomography apparatus for carrying out the computed tomography method according to the invention,

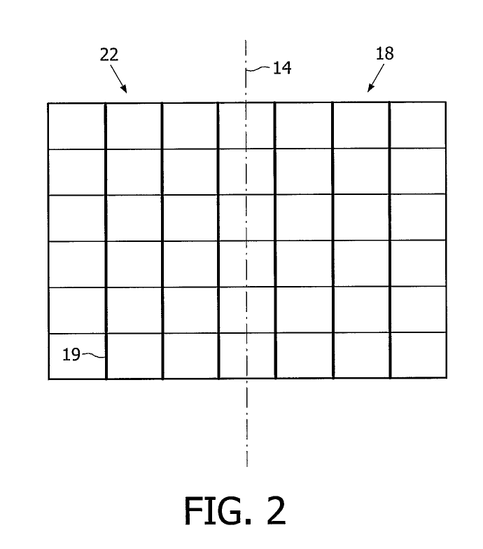

FIG. 2 shows schematically a top view of a rolled out detecting surface of a detector unit having a one-dimensional anti-scatter grid,

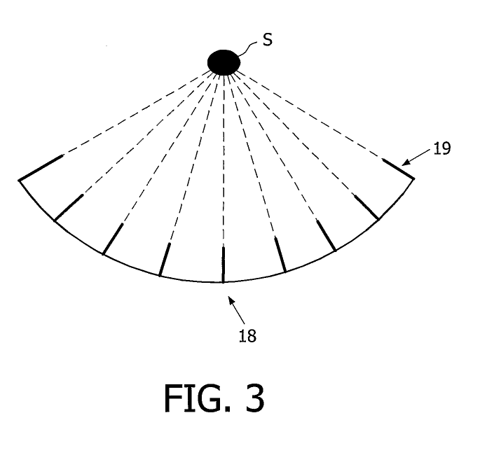

FIG. 3 shows schematically a lateral view of a radiation source and the detecting surface seen in a direction parallel to an axis of rotation of the computed tomography apparatus,

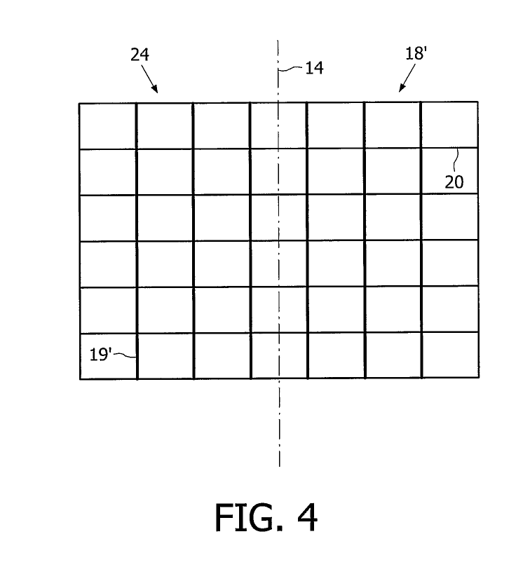

FIG. 4 shows schematically a top view of another rolled out detecting surface of a detector unit having a two-dimensional anti-scatter grid,

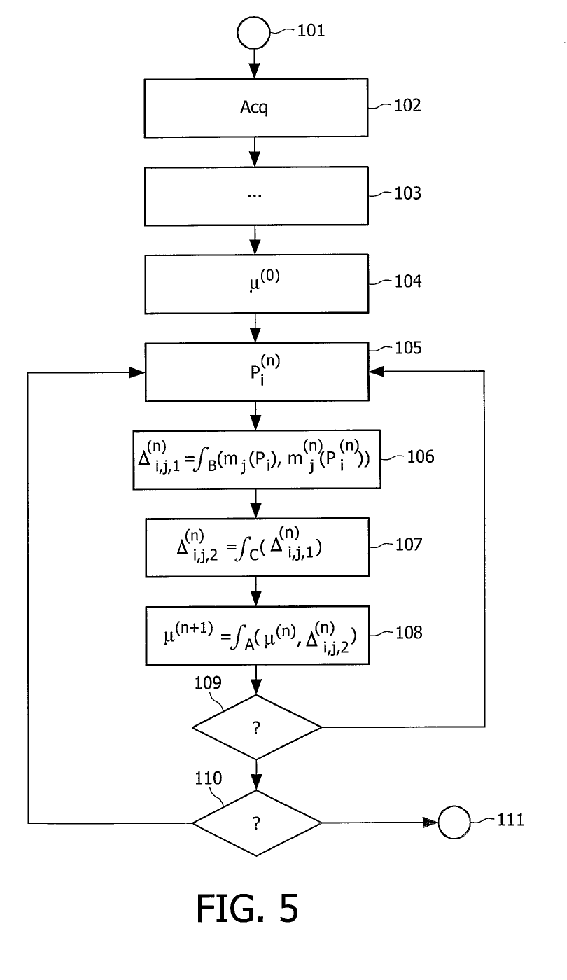

FIG. 5 shows a flow chart illustrating a computed tomography method in accordance with the invention,

FIG. 6 shows schematically a detecting surface, one focal spot position and an examination zone,

FIG. 7 shows schematically the detecting surface, two focal spot positions and the examination zone, and

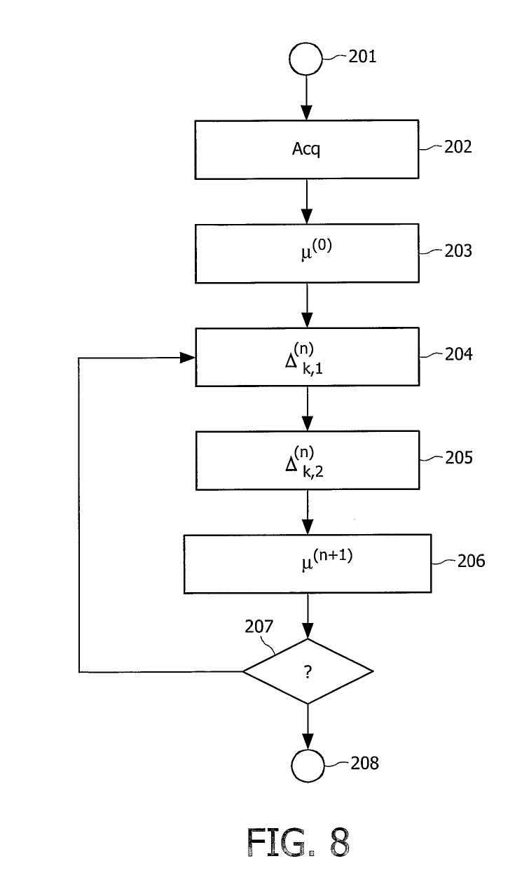

FIG. 8 shows a flow chart illustrating another computed tomography method according to the invention.

The computed tomography apparatus shown in FIG. 1 includes a gantry 1 which is capable of rotation about an axis of rotation 14 which extends in a direction parallel to the z direction of the co-ordinate system shown in FIG. 1. To this end, the gantry is driven by a motor 2 at a preferably constant but adjustable angular speed. A radiation source S, in this embodiment a x-ray source, is mounted on the gantry. The x-ray source is provided with a collimator arrangement 3 which forms a conical radiation beam 4 from the radiation produced by the radiation source S, that is, a radiation beam having a finite dimension other than zero in the z direction as well in a direction perpendicular thereto (that is, in a plane perpendicular to the axis of rotation).

In this embodiment the radiation source S is a x-ray tube capable of moving the focal spot (emitting area) parallel to the axis of rotation 14. In particular the x-ray tube is capable of switching the focal spot position parallel to the axis of rotation 14. In this embodiment the x-ray tube is capable of switching the focal spot position between two locations having a distance of 45 mm and arranged on a line parallel to the axis of rotation 14, i.e. the focal spot is either positioned at a first location or at a second location. Alternatively, the x-ray tube can switch the focal spot position between more than two locations.

The radiation beam 4 traverses an examination zone 13 in which an object, for example, a patient on a patient table (both not shown), may be present. The examination zone 13 is shaped as a cylinder. After having traversed the examination zone 13, the x-ray beam 4 is incident on a detector unit 16 with a two-dimensional detecting surface 18. The detector unit 16 is mounted on the gantry and includes a number of detector rows, each of which includes a plurality of detector elements. The detector rows are situated in planes extending perpendicularly to the axis of rotation, preferably on an arc of a circle around the radiation source S, but they may also have a different shape, for example, they may describe an arc of a circle around the axis of rotation 14 or may be straight. Each detector element struck by the radiation beam 4 delivers a measured value for a ray of the radiation beam 4 in any position of the radiation source.

FIG. 2 shows schematically a top view of a part of the rolled out detecting surface 18 of the detector unit 16. The detector unit comprises an one-dimensional anti-scatter grid 22 with lamellae 19 oriented parallel to the axis of rotation 14 and arranged on the detecting surface 18 of the detector unit 16 between adjacent detector elements.

FIG. 3 shows schematically a lateral view of the detecting surface 18 of the detector unit 16 and the radiation source S seen in a direction parallel to the axis of rotation 14. The detecting surface 18 is not rolled out in FIG. 3. As it can be seen in FIG. 3, the lamellae 19 are focus-centered relative to the focal position yielding a reduction of scattered radiation detected by the detector elements without shadowing effects.

Alternatively, the detector unit 16 could comprise a two-dimensional anti-scatter grid 24, as shown in FIG. 4. In FIG. 4 the detecting surface 18′ is rolled out and comprises lamellae 19′ oriented parallel to the axis of rotation 14 and lamellae 20 oriented perpendicular to the lamellae 19′. The aspect ratio of the lamellae 19′ is larger than the aspect ratio of the lamellae 20 wherein the aspect ratio is defined by the ratio of the height of the respective lamellae to the width of a detector element in a direction perpendicular to the respective lamellae.

Lamellae 20 oriented perpendicular to the axis of rotation 14 can only be focus-centered to one focal spot position. Since during acquisition the focal spot position is moved parallel to the axis of rotation 14, shadowing effects caused by the lamellae 20 could be substantially eliminated only for one focal spot position, but for other focal spot positions shadowing effects caused by the lamellae 20 are present. One solution to eliminate these shadowing effects is to use a one-dimensional anti-scatter grid 22 as shown in FIGS. 2 and 3. But this one-dimensional ant-scatter grid 22 has the disadvantage, that the detection of radiation scattered in the direction of the axis of rotation 14 is not reduced. Thus, the aspect ratio of the lamellae 20 is optimized such that detection of radiation scattered in a direction parallel to the axis of rotation 14 and shadowing effects in this direction are simultaneously as small as possible, i.e. the aspect ratio of the lamellae 20 is at least smaller than the aspect ratio of the lamellae 19′.

The height of the lamellae 19, 19′ and 20 is particularly some centimeters, e.g. 1, 2, 3, 4 or 5 cm.

The angle of aperture of the radiation beam 4, denoted by the reference αₘₐₓ (the angle of aperture is defined as the angle enclosed by a ray that is situated at the edge of the radiation beam 4 in a plane perpendicular to the axis of rotation relative to a plane defined by the radiation source S and the axis of rotation 14), then determines the diameter of the object cylinder in which the object to be examined is situated during acquisition of the measured values. The examination zone 13, or the object or patient table, can be displaced parallel to the axis of rotation 14 or the z axis by means of a motor 5. Equivalently, however, the gantry could also be displaced in this direction.

When the motors 5 and 2 run simultaneously, the radiation source S and the detector unit 16 describe a helical trajectory relative to the examination zone 13. This helical motion can be used for the pre-acquisition described further below. However, when the motor 5 for the displacement in the z direction is inactive and the motor 2 rotates the gantry, a circular trajectory is obtained for the motion of the radiation source S and the detector unit 16 relative to the examination zone 13. This circular motion is used during the acquisition of measured values in step 102, also described further below.

The measured values acquired by the detector unit 16 are transferred to an reconstruction unit 10 which reconstructs the absorption distribution in at least a part of the examination zone 13 for display, for example, on a monitor 11. The two motors 2 and 5, the reconstruction unit 10, the radiation source S and the transfer of the measured values from the detector unit 16 to the reconstruction unit are controlled by a control unit 7.

FIG. 5 shows the execution of a computed tomography method in accordance with the invention which can be carried out by means of the computed tomography apparatus of FIG. 1.

After the initialization in step 101 the gantry 1 rotates at a constant angular speed.

In step 102 the radiation of the radiation source S is switched on, and measured values are acquired by the detector elements of the detector unit 16. During acquisition the x-ray tube switches the focal spot between two locations arranged on a line parallel to the axis of rotation and having in this embodiment a distance of 45 mm. This distance can vary in other embodiments.

Measured values, which were detected while the radiation source was in the same angular position, are referred to as a projection. The x-ray tube switches the focal spot from projection to projection, i.e. for adjacent angular positions of the radiation source the focal spot position is different. If the x-ray tube has first and second locations, where the focal spot can be situated, and if the focal spot is situated at the first location, when the radiation source is at a certain angular position, at which measured values are detected, then the focal spot is situated at the second location, when the radiation source is at a angular position, at which measured values are detected, adjacent to the certain angular position.

Switching the focal spot from one location to the other location from projection to projection results in a good sampling in a direction parallel to the axis of rotation, and thus in an improved image quality, and enlarges the reconstructable part of the examination in this direction.

The enlargement of the reconstructable part of the examination zone is apparently by comparing FIGS. 6 and 7. In FIG. 6 an image of an object 25, e.g. a human heart, should be reconstructed and therefore a part of the examination zone is selected, e.g. by a radiologist, in which the object 25 is situated and from which an image should be reconstructed. This selected part of the examination zone is referred to as field of view (FOV). In FIG. 6 a known gantry with a focal spot is used, which is not moveable along a line 27 parallel to the axis of rotation 14, i.e. the focal spot is stationary within the radiation source S. In this arrangement some parts of the field of projection are not irradiated, or some parts are irradiated only from too few angular positions of the radiation source not allowing to reconstruct these parts. These parts might be the outer parts 29 and 31 of the field of projection which are close to the axis of rotation 14 and which are spaced apart from the plane in which the radiation source S rotates. In FIG. 7 the x-ray tube is capable of switching the focal spot position from a first location 23a to a second location 23b and reverse. With this kind of x-ray tube also the parts 29 and 31 are irradiated from enough angular positions of the radiation source allowing to reconstruct also these parts 29 and 31 and thus the whole field of view.

For reconstruction the field of view is divided into voxels. It is well known, that a voxel is reconstructable, if it is irradiated from radiation beams which are distributed over an angular range of at least 180°. In the arrangement of FIG. 6 the voxel situated in the parts 29 and 31 of the field of projection are not irradiated over an angular range of at least 180°. Thus, these parts are not reconstructable. In the arrangement of FIG. 7 in accordance with the invention also the parts 29 and 31 are irradiated over an angular range of at least 180°, so that the whole field of view is reconstructable. Thus, in contrast to a stationary focal spot, as shown in FIG. 6, the field of view can be increased.

In other embodiments, if an image of a heart has to be reconstructed, an electrocardiograph measures an electrocardiogram during acquisition and transfers the electrocardiogram to the control unit 7. The control unit 7 controls the radiation source S such that the radiation is switched off, if the heart is moving faster and that the radiation source is switched on, if the heart is moving slower during each cardiac cycle. Other known, so-called gating techniques, can also be used to modulate the intensity of the radiation emitted by the radiation source S depending on the heart motion. These gating techniques are, e.g., disclosed in “Cardiac Imaging with X-ray Computed Tomography: New Approaches to Image Acquisition and Quality Assurance”, Stefan Ulzheimer, Shaker Verlag, Germany, ISBN 3-8265-9302-2.

Furthermore, the tube current of the x-ray source, i.e. of the radiation source, can be modulated depending on the diameter of the object in different directions. For example, if an image of a human patient has to be reconstructed and the patient lies on his back, the diameter of the patient in a horizontal direction is larger than in a vertical direction. Thus, the tube current and therefore the intensity of the radiation beam is modulated in a way, that it is larger in a horizontal direction than in a vertical direction.

In the following steps an image of the examination zone is reconstructed iteratively. Here, the algebraic reconstruction technique (ART) is used. Alternatively, other known iterative reconstruction methods, e.g. the maximum likelihood method, can be used.

In step 103 a sequence is provided in which the different projections are considered during reconstruction. The sequence is a random sequence, but the reconstruction in the scope of the invention is not limited to a random sequence. Alternatively, the sequence might be, e.g., a successive sequence in which projections, which have been measured successively, are considered successively. Furthermore, some projections might be discarded or weighted. If an image of a moving object, as a human heart, has to be reconstructed, projections, which were measured while the object was in a faster moving phase in each cardiac cycle, could be discarded or multiplied by a smaller weighting factor, and projections, which were measured while the object was in a slower moving phase, could be considered in the sequence and multiplied by a larger weighting factor. This weighting or discarding of projections depending on the heart motion is discussed in more in detail in the above mentioned “Cardiac Imaging with X-ray Computed Tomography: New Approaches to Image Acquisition and Quality Assurance”, Stefan Ulzheimer, Shaker Verlag, Germany, ISBN 3-8265-9302-2.

In the case of a heart, the moving phase could be detected by a electrocardiograph during the acquisition of the measured values, which transfers the measured electrocardiogram to the reconstruction unit 10.

In step 104 a field of view is selected, e.g. by a radiologist, which includes the object which has to be reconstructed. Furthermore, an initial image μ⁽⁰⁾ of this field of view is provided. The initial image μ⁽⁰⁾ is an zero image consisting of voxels with initial values zero. Alternatively, a pre-acquisition can be carried out and an initial image can be reconstructed from measured values of this pre-acquisition. During the pre-acquisition the radiation source moves, with stationary or moving focal spot, on a helical trajectory relative to the field of view in a way that at least a part of the field of view is reconstructable with known reconstruction methods, like the filtered back projection method. During the pre-acquisition the intensity of the radiation beam is lower than during the acquisition of step 102. The pre-acquisition can be carried out before or after step 102. This pre-acquisition and the reconstruction using measured values of the pre-acquisition is disclosed in U.S. Pat. No. 6,480,561.

The reconstructed initial image, which has been reconstructed using the measured values of the pre-acquisition, is interpolated to the size of the field of view and to the resolution of the final image of the field of view, and this initial image is smoothed to remove high frequency components. Using a initial image of this kind leads to strongly reduced artifacts at the borders of the field of view.

In step 105 the first measured projection Pᵢ is selected from the sequence provided in step 103. If not all projections have been considered with the same frequency, the measured projection Pᵢ is selected which follows the projection considered last. Furthermore, a projection Pᵢ⁽ⁿ⁾ is calculated by forward projection through initial image μ⁽⁰⁾ along the beams generating the measured values mⱼ(Pᵢ) of the measured projection Pᵢ, wherein mⱼ(Pᵢ) is the j-th measured value of the i-th measured projection. If a intermediate image μ⁽ⁿ⁾ has already been calculated in step 108, then the forward projection is carried out through the intermediate image μ⁽ⁿ⁾ calculated last.

The forward projection is well known. In a simple way, a calculated value mⱼ⁽ⁿ⁾(Pᵢ⁽ⁿ⁾) of the calculated projection Pᵢ⁽ⁿ⁾ can be determined by adding the values of all voxels through which the beams run which have generated the corresponding measured value mⱼ(Pᵢ) of the corresponding measured projection Pᵢ. Here mⱼ⁽ⁿ⁾(Pᵢ⁽ⁿ⁾) is the j-th calculated value of the i-th calculated projection.

In step 106 for each measured value mⱼ(Pᵢ) of the measured projection Pᵢ a disagreement value Δ_{i,j,1}⁽ⁿ⁾=ƒ_{B}(mⱼ(Pᵢ), mⱼ⁽ⁿ⁾(Pᵢ⁽ⁿ⁾)) is calculated, which is a measure for the disagreement of the measured value mⱼ(Pᵢ) from the corresponding calculated value mⱼ⁽ⁿ⁾(Pᵢ⁽ⁿ⁾) of the corresponding calculated projection Pᵢ⁽ⁿ⁾. This disagreement value is calculated using a disagreement function ƒ_{B}. In this embodiment the disagreement function is the difference of the respective calculated value mⱼ⁽ⁿ⁾(Pᵢ⁽ⁿ⁾) and the corresponding measured value mⱼ(Pᵢ) of the projections Pᵢ and Pᵢ⁽ⁿ⁾, respectively, i.e. each calculated value mⱼ⁽ⁿ⁾(Pᵢ⁽ⁿ⁾) of the calculated projection Pᵢ⁽ⁿ⁾ is subtracted from the corresponding measured value mⱼ(Pᵢ) of the measured projection Pᵢ.

In step 107 each disagreement value is weighted by a weighting function ƒ_{C}. The weighting function defines the degree of contribution of the disagreement values to the image. In this embodiment the weighting function is a weighting factor between zero and two. Thus, each disagreement value Δ_{i,j,1}⁽ⁿ⁾ is multiplied by the weighting factor.

The weighted disagreement values Δ_{i,j,2}⁽ⁿ⁾ are back projected in step 108 in the field of view along the corresponding beams of the measured projection Pᵢ modifying the intermediate image μ⁽ⁿ⁾. If the step 108 is carried out for the first time, the back projection modifies the initial image μ⁽⁰⁾. The result of the back projection is the intermediate image μ⁽ⁿ⁺¹⁾=ƒ_{A}(μ⁽ⁿ⁾,Δ_{i,j,2}⁽ⁿ⁾), wherein the function ƒ_{A} describes the back projection.

Also the back projection is well known. In a simple way, a weighted disagreement value Δ_{i,j,2}⁽ⁿ⁾ is back projected by determining the voxels of the field of view, through which the beams run, which generated the measured value mⱼ(Pᵢ), from which the corresponding calculated value mⱼ⁽ⁿ⁾(Pᵢ⁽ⁿ⁾) has been subtracted to achieve the corresponding disagreement value Δ_{i,j,1}⁽ⁿ⁾. Then the weighted disagreement value Δ_{i,j,2}⁽ⁿ⁾ is divided by the number of the determined voxels, and this divided value is added on each of the determined voxels.

In step 109 it is checked, whether each of the projections of the sequence provided in step 103 have been considered with the same frequency. If this is the case, the computed tomography method continues with step 110. Otherwise, step 105 follows.

In step 110 it is checked, whether a terminating condition is fulfilled. If this is the case, the computed tomography method ends in step 111, wherein the current intermediate image μ⁽ⁿ⁺¹⁾ is the final reconstructed image of the field of view. Otherwise, the computed tomography method continues with step 105 starting with the first projection of the sequence provided in step 103.

The terminating condition is fulfilled, if steps 105 to 109 have been carried out a predetermined number of times. Alternatively, the terminating condition is fulfilled, if the square deviation of the calculated values of the calculated projections from the measured values of the measured projections are smaller than a predetermined threshold, i.e. for example

$${\sum }_{i , j} {( {m}_{j} ( {P}_{i} ) - {m}_{j}^{( n )} ( {P}_{i}^{( n )} ) )}^{2} < t , \qquad ( 1 )$$

wherein t is the threshold.

As mentioned above, instead of the algebraic reconstruction technique described with reference to the steps 104 to 110 the maximum likelihood method could be used.

FIG. 8 shows the execution of another embodiment of the computed tomography method in accordance with the invention which can be carried out by means of the computed tomography apparatus of FIG. 1 and which uses the maximum likelihood method.

After initialization in step 201 the gantry 1 rotates at constant angular speed.

In step 202 the radiation of the radiation source is switched on, and measured values are acquired by the detector elements of the detector unit 16 as described above with reference to step 102.

In step 203 a field of view is selected, e.g. by a radiologist, which includes the object which has to be reconstructed. Furthermore, an initial image μ⁽⁰⁾ of this field of view is provided as described above with reference to step 104.

In step 204 for each voxel of the field of view a disagreement value Δ_{k,1}⁽ⁿ⁾ is calculated using following equation:

$${Δ}_{k , 1}^{( n )} = {\sum }_{u = 1}^{{N}_{y}} {a}_{u , k} ( 1 - \frac{{y}_{u}}{{b}_{u} {e}^{- {l}_{u}^{( n )}} + {r}_{u}} ) {b}_{u} {e}^{- {l}_{u}^{( n )}} , \qquad ( 2 )$$

wherein N_{y} is the overall number of measured values, i.e. the product of the number of radiation source positions during acquisition and the number of detector elements. Furthermore, a_{u,k} is a weighting factor associated with the u-th measured value and the k-th voxel, yᵤ is the number of photons which generated the u-th measured value, bᵤ is the number of photons emitted from the focal spot in the direction pointing from the focal spot position associated with the u-th measured value to the position of the center of the detector element associated with the u-th measured value during the acquisition of the u-th measured value, rᵤ is a random value contributing to the u-th measured value and lᵤ⁽ⁿ⁾ is a line integral through the field of view, i.e. through the intermediate image μ⁽ⁿ⁾ of the field of view along a ray running from the focal spot position associated with the u-th measured value to the position of the center of the detector element associated with the u-th measured value, i.e. along the ray associated with the u-th measured value.

The weighting factor a_{u,k} describes the contribution of the k-th voxel to the u-th measured value, if all voxels would have the same absorption value μₖ⁽ⁿ⁾, wherein μₖ⁽ⁿ⁾ is the absorption value of the k-th voxel after n iterations. The factor a_{u,k} is well known and depends on the used forward and back projection model. In a simple model, during forward projection all absorption values belonging to voxels transmitted by the ray associated with the u-th measured value are added to get a calculated measured value. In this simple forward projection model a weighting factors a_{u,k} is equal to one, if the ray associated with the u-th measured value transmits the k-th voxel, and otherwise a_{u,k} is equal to zero. Alternatively, other known forward and back projection models might be used yielding other weighting factors, e.g. forward and back projection models using spherical base functions instead of voxels (so called “blobs”).

In order to get the number of photons yᵤ, which generated the u-th measured value, a detector unit can be used, which directly measures this number of photons yᵤ. Alternatively, if the detector unit 16 is used, which measures values vᵤ depending on the intensity, the number of photons yᵤ can be calculated from measured values vᵤ using yᵤ=bᵤe^{-v}^{ᵤ}, wherein the number of photons bᵤ can be measured by acquiring measured values according to step 202 without an object in the examination zone and by calculating the number of photons bᵤ from the measured values without an object using the photon spectrum. This kind of calculation is well known and will therefore not be explained in detail. Furthermore, the number of photons bᵤ is a system parameter of the computed tomography apparatus and is normally known.

If the acquired values are measured values vᵤ depending on the intensity and if the radiation source emits radiation isotropicly in the direction of each detector element, i.e. if all bᵤ are equal, the equation (2) and the equations (3) and (4) described below can be transformed to an equation (5) allowing to use directly the measured values vᵤ for reconstruction.

The random value rᵤ contributing to the u-th measured value is generally generated by scattered rays. In this embodiment a one-dimensional 22 or two-dimensional anti-scatter grid 24 is used so that random values can be neglected in the following.

The line integral lᵤ⁽ⁿ⁾ through the intermediate image μ⁽ⁿ⁾ along the ray associated with the u-th measured value describes a forward projection. Thus, this line integral is lᵤ⁽ⁿ⁾ is well known and depends on the used forward projection model. In the above explained simple forward projection model the line integral lᵤ⁽ⁿ⁾ is the sum of all absorption values belonging to voxels transmitted by the ray associated with the u-th measured value. If another forward projection model is used, the line integral lᵤ⁽ⁿ⁾ has to be modified accordingly.

After disagreement values Δ_{k,1}⁽ⁿ⁾ have been calculated for each voxel, in step 205 each disagreement value Δ_{k,1}⁽ⁿ⁾ is weighted according to following equation:

$${Δ}_{k , 2}^{( n )} = \frac{{Δ}_{k , 1}^{( n )}}{{\sum }_{u = 1}^{{N}_{y}} {a}_{u , k} {a}_{u} {c}_{u}^{( n )}} . \qquad ( 3 )$$

Here Δ_{k,2}⁽ⁿ⁾ is the weighted disagreement value and aᵤ is equal to

$${\sum }_{k} {a}_{u , k} ,$$

i.e. aᵤ is the sum over all weighting factors a_{u,k} for voxels, which contribute to the u-th measured value. Furthermore, cᵤ⁽ⁿ⁾ is the curvature associated with the u-th measured value and the intermediate image μ⁽ⁿ⁾. The curvature and the whole maximum likelihood method is well known and in more detail described in the “Handbook of Medical Imaging”, Volume 2, 2000, by Milan Sonka and J. M. Fitzpatrick.

Here, the curvature is given by

$${c}_{u}^{( n )} = {b}_{u} {e}^{- {l}_{u}^{( n )}} . \qquad ( 4 )$$

Inserting equation (4) in equation (3), inserting equation (3) in equation (2), neglecting the random value rᵤ, considering yᵤ=bᵤe^{-v}^{ᵤ} and assuming an isotropicly emitting radiation source, i.e. b=bᵤ leads to:

$${Δ}_{k , 2}^{( n )} = \frac{{\sum }_{u = 1}^{{N}_{y}} {a}_{u , k} ( {e}^{- {l}_{u}^{( n )}} - {e}^{- {v}_{u}} )}{{\sum }_{u = 1}^{{N}_{y}} {a}_{u , k} {a}_{u} {e}^{- {l}_{u}^{( n )}}} . \qquad ( 5 )$$

Thus, instead of calculating the disagreement Δ_{k,1}⁽ⁿ⁾ according to equation (2) in step 204 and the weighted disagreement value according to equation (3) in step 205, the weighted disagreement value can be directly calculated using equation (5) and measured values vᵤ, which depend on the intensity and which have seen acquired by the detector unit 16.

In step 206 the intermediate image μ⁽ⁿ⁾ is updated according to the following equation:

$${μ}_{k}^{( n + 1 )} = [ {μ}_{k}^{( n )} + {Δ}_{k , 2}^{( n )} ] . \qquad ( 6 )$$

The expression [x]₊ describes that x is set to zero, if x is smaller than zero, and otherwise x is not modified.

According to equation (6) in step 206 for each k-th voxel the weighted disagreement value Δ_{k,2}⁽ⁿ⁾ for the k-th voxel is added to the intermediate absorption value μₖ⁽ⁿ⁾ of the k-th voxel resulting in an updated absorption value μₖ⁽ⁿ⁺¹⁾ for the k-th voxel.

In step 207 it is checked, whether a terminating condition is fulfilled. If this is the case, the computed tomography method ends in step 208, wherein the current intermediate image μ⁽ⁿ⁺¹⁾ is the final reconstructed image of the field of view. Otherwise, the computed tomography method continues with step 204.

The terminating condition is fulfilled, if steps 204 to 206 have been carried out a predetermined number of times. Alternatively, other known termination conditions can be used. For example, the terminating condition could be fulfilled, if the square deviation of the calculated line integrals lᵤ⁽ⁿ⁾ from the associated measured values vᵤ is smaller than a predetermined threshold.

1. A computed tomography method comprising the steps of:
generating a circular relative motion between an examination zone and a radiation source about an axis of rotational,
generating a conical radiation beam using the radiation source, wherein the conical radiation beam is emitted from an emitting area of the radiation source, wherein the conical radiation beam traverses the examination zone and wherein the position of the emitting area is moved parallel to the axis of rotation during the relative motion,
acquiring measured values using a detector unit during the relative motion, wherein the measured values depend on the intensity of the conical radiation beam after traversing the examination zone,
switching the position of the emitting area between at least two positions spaced apart from each other and arranged on a line parallel to the axis of rotation during the relative motion,
reconstructing an image of the examination zone using the measured values.

2. The computed tomography method according to claim 1, wherein during the relative motion the radiation source runs through different radiation source positions relative to the examination zone, wherein in each of the radiation source positions the measured values are acquired and wherein the position of the emitting area, while the radiation source is in a radiation source position, is different from the position of the emitting area, while the radiation source is in a consecutive radiation source position.

3. The computed tomography method according to claim 1 wherein the image of the examination zone is reconstructed using an iterative reconstruction method, in particular an algebraic reconstruction method or a maximum likelihood method.

4. A computed tomography apparatus comprising:
a drive arrangement for generating a circular relative motion between an examination zone and a radiation source about an axis of rotation,
a radiation source for generating a conical radiation beam for traversing the examination zone, wherein the radiation source comprises an emitting area from which the conical radiation beam is emitted and wherein the position of the emitting area is moveable parallel to the axis of rotation during the relative motion,
a detector unit for acquiring measured values during the relative motion,
a reconstruction unit for reconstructing an image of the examination zone using the measured values,
a control unit for controlling of the drive arrangement, the radiation source, the detector unit and the reconstruction unit according to the steps of claim 1.

5. The computed tomography apparatus according to claim 4, wherein the detector unit comprises a one-dimensional anti-scatter grid with lamellae being oriented parallel to the axis of rotation.

6. The computed tomography apparatus according to claim 4, wherein the detector unit comprises a two-dimensional anti-scatter grid with lamellae being oriented parallel to the axis of rotation and with lamellae being oriented perpendicular to the axis of rotation wherein the aspect ration of the lamellae being oriented parallel to the axis of rotation is larger than the aspect ration of the lamellae being oriented perpendicular to the axis of rotation.

7. A computer program for a control unit for controlling a drive arrangement, a radiation source, a detector unit and a reconstruction unit of a computed tomography apparatus according to the steps of claim 1.
